(19) United States
(12) Patent Application Publication
(10) Pub. No.: US 2022/0361978 A1
(43) Pub. Date: Nov. 17, 2022

(54) ENDOSCOPE CLEANING DEVICE AND CLEANING METHOD THEREOF

(71) Applicant: ADRONIC ENDOSCOPE CO., LTD., Taichung City (TW)

(72) Inventors: HSIANG-TE TSENG, Taichung City (TW); YEN-WEN CHEN, Taichung City (TW); SHIH-HONG HUANG, Taichung City (TW)

(73) Assignee: ADRONIC ENDOSCOPE CO., LTD., Taichung City (TW)

(21) Appl. No.: 17/317,509

(22) Filed: May 11, 2021

(51) Int. Cl. A61B 90/70; A61B 1/12; A61B 1/00

(52) U.S. Cl. CPC A61B 90/70; A61B 1/12; A61B 1/00002; A61B 2090/701

(57) ABSTRACT

An endoscope cleaning device is applied to an endoscope having a main body and a tube body connected to the main body. The endoscope cleaning device includes a frame, a receiving tank for receiving the tube body, a circulating module, and an operating module that is electrically connected to the circulating module. The receiving tank and the operating module are disposed on the frame. The circulating module is connected to a source of first solution supply, a source of second solution supply, and the receiving tank. The operating module is manipulated by a user to control the circulating module to transmit the solution provided by the source of first solution supply and the solution provided by the source of second solution supply to the receiving tank and then to drain the solution in the receiving tank out. A cleaning method of an endoscope by using the endoscope cleaning device is provided.

### BACKGROUND OF THE INVENTION

### Technical Field

The present invention relates generally to an endoscope, and more particularly to an endoscope cleaning device and a method for cleaning an endoscope.

### Description of Related Art

Nowadays, to avoid infection, most of the cheap medical supplies, such as syringes and injection equipment are disposable. However, some expansive medical supplies, such as a fiberscope, a gastroscope, a celoscope, a thoracoscope, an arthroscope, and other endoscopes and the supplies of the endoscope, such as a catheter or a tube, need to be used repeatedly in normal. Additionally, since the endoscope and the supplies of the endoscope are adapted to be inserted into a human body to touch the damaged skin or mucosa. Therefore, after the endoscope is used, the endoscope needs to be cleaned, disinfected, and sterilized, thereby avoiding cross-infection.

A process of cleaning the catheter or the tube of the endoscope is complicated. Except for washing with detergent, the catheter and the tube of the endoscope still need to be completed washed with water to remove contaminants and detergent. Besides, to completely perform the cleaning effect of the detergent, a time to sink the endoscope in the detergent should be sufficient and precisely controlled, so that how to set up a simple standardized cleaning process is a problem that needs to be solved.

### BRIEF SUMMARY OF THE INVENTION

In view of the above, the primary objective of the present invention is to provide an endoscope cleaning device and an endoscope cleaning method, which could effectively enhance cleaning efficiency and quality.

The present invention provides an endoscope cleaning device that is applied to an endoscope. The endoscope includes a main body and a tube body which is connected to the main body. The endoscope cleaning device includes a frame, a receiving tank, a circulating module, and an operating module. The receiving tank is disposed on the frame and is adapted to receive the tube body of the endoscope. The circulating module is connected to a source of first solution supply, a source of second solution supply, and the receiving tank. The operating module disposed on the frame and electrically connected to the circulating module, wherein the operating module is provided for a user to control the circulating module to respectively transmit a solution provided from the source of first solution supply and a solution provided from the source of second solution supply to the receiving tank, and to drain the solution in the receiving tank out of the receiving tank.

The present invention provides a cleaning method of an endoscope including: A. putting the tube body of the endoscope into the receiving tank; and B manipulating the operating module to control the circulating module to initiate an automatic cleaning process, wherein the automatic cleaning process includes at least one disinfecting step and at least one cleansing step in order. The at least one disinfecting step includes transmitting a solution of the source of first solution supply to the receiving tank to allow the tube body to sink into the solution outputted from the source of first solution supply, and then discharge the solution in the receiving tank out of the receiving tank. The at least one cleansing step includes transmitting a solution of the source of second solution supply to the receiving tank to allow the tube body to sink into the solution outputted from the source of second solution supply, and then drain the solution in the receiving tank out of the receiving tank.

By using the endoscope cleaning device, the user could operate the operating module to control the circulating module to transmit the solution of the source of first solution supply and the solution of the source of second solution supply to the receiving tank and to drain the solution in the receiving tank by a predetermined order. Therefore, the automatic cleaning process could be conducted to enhance the cleaning efficiency and quality.

### BRIEF DESCRIPTION OF THE SEVERAL VIEWS OF THE DRAWINGS

The present invention will be best understood by referring to the following detailed description of some illustrative embodiments in conjunction with the accompanying drawings, in which

FIG. 1 is a perspective view of the endoscope cleaning device of an embodiment according to the present invention;

FIG. 2 is a perspective view of the endoscope cleaning device of the embodiment according to the present invention, showing the endoscope is disposed on the endoscope cleaning device;

FIG. 3 is a schematic view of the endoscope cleaning device of the embodiment according to the present invention;

FIG. 4 is a side view of the endoscope cleaning device of the embodiment according to the present invention;

FIG. 5 is a sectional view taken along the 5-5 line in FIG. 4;

FIG. 6 is a schematic diagram of the endoscope cleaning device of the embodiment of the present invention; and,

FIG. 7 is a flowchart of the method of cleaning the endoscope of an embodiment of the current application.

### DETAILED DESCRIPTION OF THE INVENTION

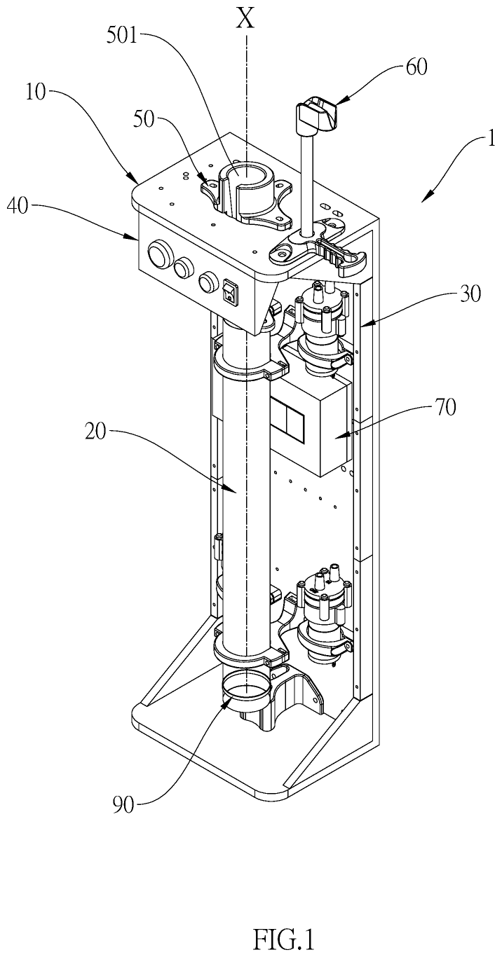

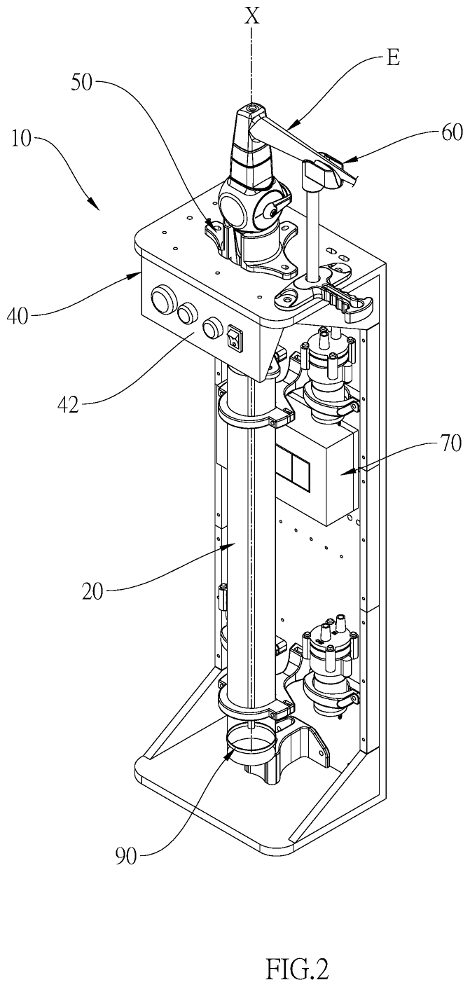

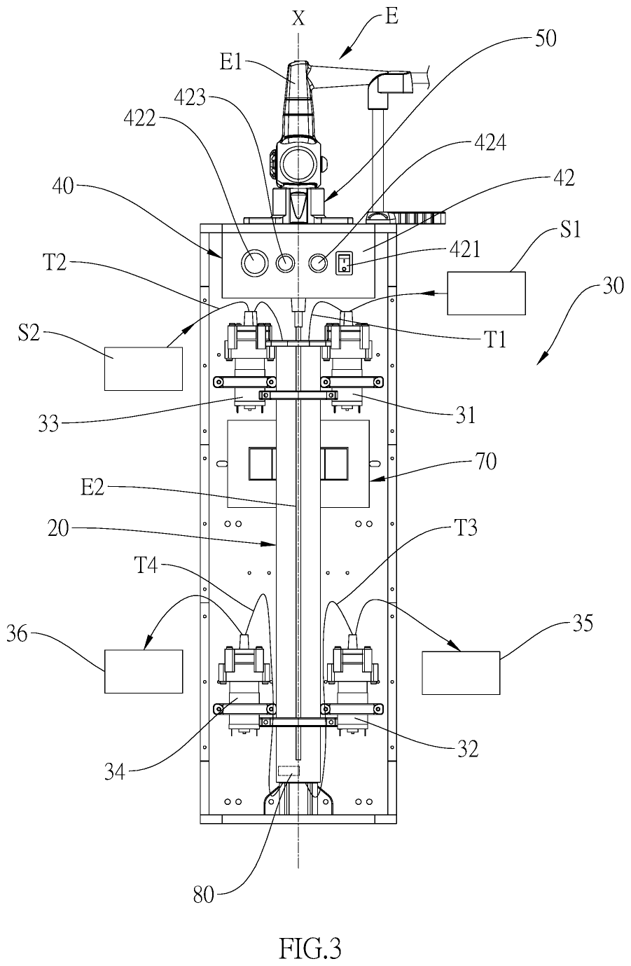

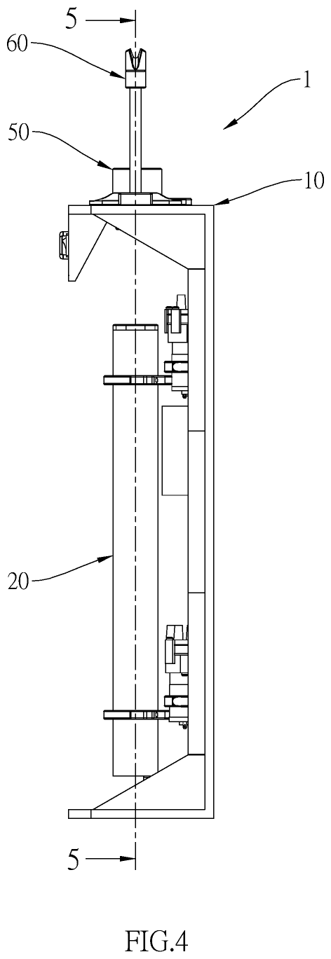

As illustrated in FIG. 1 to FIG. 5 an endoscope cleaning device 1 of an embodiment of the present invention is adapted to clean an endoscope E, wherein the endoscope E includes a main body E1 and a tube body E2 which is connected to the main body E1. The tube body E2 is a flexible tube that is adapted to be inserted into a human body for inspecting. In the current embodiment, the tube body E2 is a tube having flexibility as an example. However, in other embodiments, the tube body E2 could be other types or be made of other materials, such as a hard tube body made of stainless steel.

The endoscope cleaning device 1 includes a frame 10, a receiving tank 20, a circulating module 30, and an operating module 40. The receiving tank 20 is disposed on the frame 10 and is adapted to receive the tube body E2 of the endoscope E. The receiving tank 20 is long cylindrical, and a top end of the receiving tank 20 has an opening O. A minimum distance between the opening O and a bottom of the receiving tank 20 is greater than a length of the tube body E2, so that when the tube body E2 is received in the receiving tank 20, the tube body E2 could be sufficiently extended to avoid the tube E2 to be folded or overlapped. Therefore, the problem that a folded site or an overlapped site of the tube body E2 is hard to be cleaned could be solved or be alleviated. In other embodiments, the minimum distance could be smaller than or equal to the length of the tube body E2.

The main body E1 has a boot end and a cord end, wherein the boot end is near to the tube body E2, and the cord end is away from the tube body E2. More specifically, the boot end is connected to the tube body E2, and the cord end is connected to an umbilical cord. The endoscope cleaning device 1 includes a boot end positioning member 50 for receiving the boot end of the endoscope E, wherein the boot end positioning member 50 is disposed on the frame 10 and is located above the receiving tank 20. The boot end positioning member 50 has a through hole 501. The tube body E2 of the endoscope E could pass through the through hole 501 and the opening O of the receiving tank 20 to be received into the receiving tank 20, and the main body E1 could abut against a side of the boot end positioning member 50. With such design, the endoscope E could firmly disposed on the endoscope cleaning device 1. Besides, the receiving tank 20 has a longitudinal axis X which is parallel to a length direction of the receiving tank 20. The longitudinal axis X passes through the opening O and the through hole 501, so that a user could easily insert the tube body E2 of the endoscope E through the through hole 501 and the opening O of the receiving tank 20, and the endoscope E could be quickly aligned and received in the receiving tank 20.

The endoscope cleaning device 1 includes a cord end positioning member 60 for receiving the cord end of the endoscope E. The cord end positioning member 60 is disposed on the frame 10 and is located above the receiving tank 20. The cord end positioning member 60 is adapted to receive the cord end of the main body E1. When the boot end of the main body E1 abuts against the side of the boot end positioning member 50, and the cord end of the main body E1 is disposed in the cord end positioning member 60, the cord end positioning member 60 could restrict the main body E1 to rotate about the longitudinal axis X. With such design, the endoscope E could be firmly disposed on the endoscope cleaning device 1, thereby preventing the endoscope E from being disengaged from the endoscope cleaning device 1 when the endoscope E is hit.

The circulating module 30 is connected to a source of first solution supply S1, a source of second solution supply S2, and the receiving tank 2, respectively. The circulating module includes a first pump 31, a second pump 32, a third pump 33, a fourth pump 34, a first storage tank 35, and a second storage tank 36. The first pump 31, the second pump 32, the third pump 33, and the fourth pump 34 are disposed to the frame 10 and are controlled by the operating module 40, wherein the first pump 31 is adapted to transmit a solution of the source of first solution supply S1 to the receiving tank 20, and the second pump 32 is adapted to transmit the solution which comes from the source of first solution supply S1 in the receiving tank 20 to the first storage tank 35. The third pump 33 is adapted to transmit a solution of the source of second solution supply S2 to the receiving tank 20, and the fourth pump 34 is adapted to transmit the solution which comes from the source of second solution supply S2 in the receiving tank 20 to the second storage tank 36.

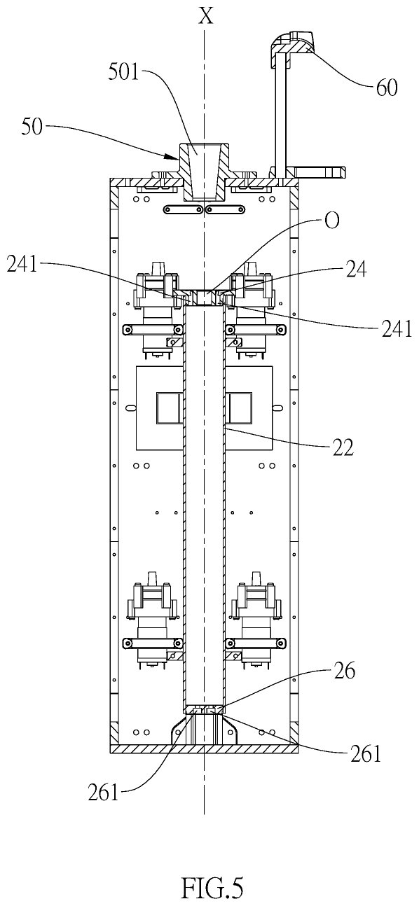

As illustrated in FIG. 5, the receiving tank 20 includes a tank body 22, a top lid 24, and a bottom lid 26. The tank body 22 is made of a transparent material (such as glass), so that the user could clearly observe a cleaning state of the tube body E2 of the endoscope E. The tank body 22 is in a tube shape, and each of two ends of the tank body 22 has an opening. Each of the top lid 24 and the bottom lid 26 are detachably engaged with one of the openings of the tank body 22 at two ends, so that the openings at the two ends of the tank body 22 are beneficial for the user to detach and clean. The top lid 24 has the opening O and two perforations 241. The bottom lid 26 has two perforations 261. One of the two perforations 241 of the top lid 24 is connected to the source of first solution supply S1 via a tube T1, and the other one of the two perforations 241 of the top lid 24 is connected to the source of second solution supply S2 via another tube T2. One of the two perforations 261 of the bottom lid 26 is connected to the first storage tank 35 via a tube T3, and the other one of the two perforations 261 of the bottom lid 26 is connected to the second storage tank 36 via another tube T4. Therefore, the first pump 31 could transmit a solution from the source of first solution supply S1 to the receiving tank 20 via the tube T1, and the second pump 32 could transmit the solution in the receiving tank 20 to the first storage tank 35 via the tube T2. The third pump 33 could transmit a solution from source of second solution supply S2 to the receiving tank 20 via the tube T2, and the fourth pump 34 could transmit the solution in the receiving tank 20 to the second storage tank 36 via the tube T4.

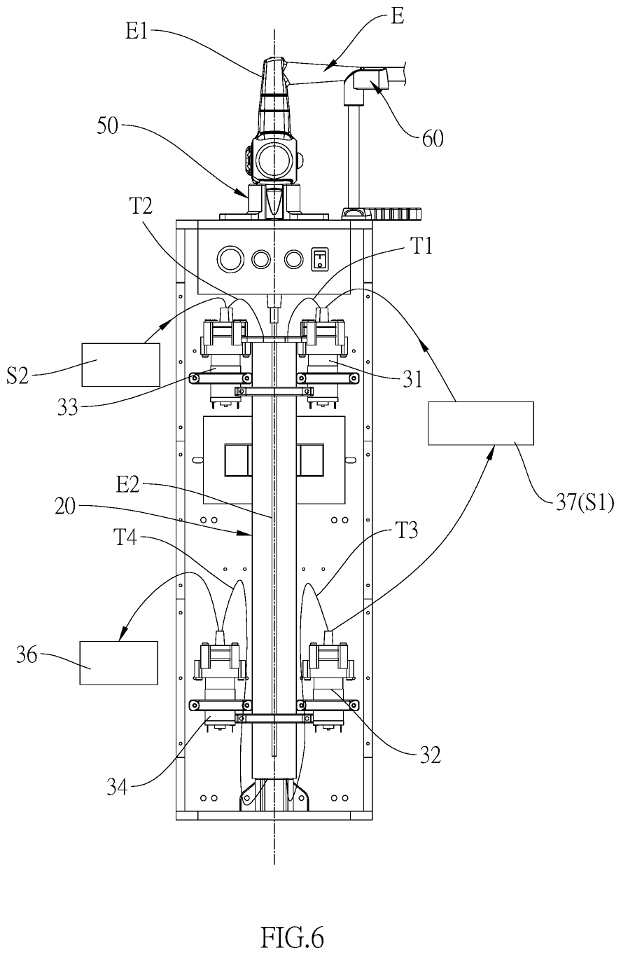

The endoscope cleaning device 1 includes a display screen 70 which is disposed on the frame 10 and is electrically connected to the operating module 40. The display screen 70 is adapted to display a time that needs for an automatic cleaning process. The operating module 40 is disposed on the frame and is electrically connected to the circulating module 30, wherein the operating module 40 includes an operating panel 42. The operating panel 42 is disposed with a power button 421, an initiate button 422, a button 423 for compulsorily draining disinfecting solution, and a button 424 for compulsorily draining water. The operating panel 42 is provided for the user to control the first pump 31, the second pump 32, the third pump 33, and the fourth pump 34 to sequentially conduct the following processes: the first pump 31 pumps a solution of the source of first solution supply S1 into the receiving tank 20; the second pump 32 pumps the solution in the receiving tank 20 to the first storage tank 35; the third pump 33 pumps a solution of the source of second solution supply S2 into the receiving tank 20; and, the fourth pump 34 pumps the solution in the receiving tank 20 to the second storage tank 36. The source of first solution supply S1 is a disinfection solution supply source. The source of second solution supply S2 is a water supply source, wherein the water could be deionized water, distilled water, filtered water, or sterile water. In the current embodiment, the source of first solution supply S1 and the first storage tank 35 could be independently disposed. In others embodiment, the source of first solution supply S1 and the first storage tank 35 could be integrally formed as the same storage tank 37, as illustrated in FIG. 6. With such design, a disinfection solution stored in the storage tank 37 could be transmitted to the receiving tank 20 by the first pump 31, and the disinfection solution in the receiving tank 20 could be transmitted back to the storage tank 37 by the second pump 32, thereby recycling the disinfection solution for being used repeatedly.

The endoscope cleaning device 1 includes an ultrasonic vibrator 80 which is located in the receiving tank 20. For example, the ultrasonic vibrator 80 could be disposed on an inner wall or a bottom surface of the receiving tank 20 to apply ultrasonic vibration to a fluid in the receiving tank 20, so that an outer surface and an inner surface of the tube body E2 could be sufficiently and completely cleaned, sterilized, and disinfected. In other embodiments, the receiving tank 20 could be disposed with a stirrer to stir the fluid in the receiving tank 20, thereby accelerating a decontaminating process of the tube body E2, namely making contaminants be disengaged from the inner surface or the outer surface of the tube body E2 faster.

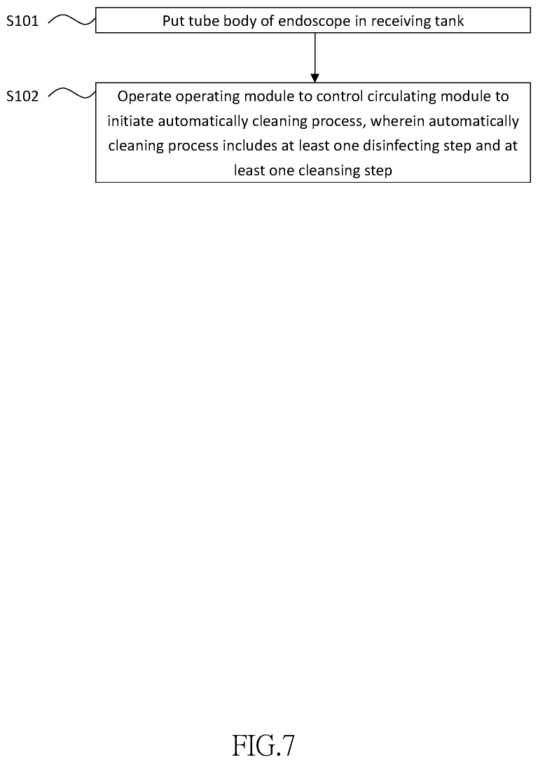

Referring to FIG. 7 which is a flow chart of a cleaning method of an embodiment according to the present invention by using the endoscope cleaning device 1 to clean the endoscope E, wherein the cleaning method includes the following steps:

Step S101: put the tube body E2 of the endoscope E into the receiving tank 20. Referring to FIG. 5, the user could insert a distal end of the tube body E2 of the endoscope E through the through hole 501 of the boot end positioning member 50 and the opening O of the receiving tank 20 to move toward the bottom of the receiving tank 20 along the longitudinal axis X of the receiving tank 20. With such design, the tube body E2 of the endoscope E could pass thorough the through hole 501 and the opening O of the receiving tank 20 to be received in the receiving tank 20, and the main body E1 of the endoscope E could abuts against the side of the boot end positioning member 50. Besides, a part of the main body E1 could be disposed on the cord end positioning member 60, so that the main body E1 could be restricted by the cord end positioning member 60 to rotate about the longitudinal axis X. Therefore, the endoscope E could be firmly disposed on the endoscope cleaning device 1. Even though the endoscope E is hit, the endoscope E is hard to be disengaged from the endoscope cleaning device 1.

Step S102: manipulate the operating module 40 to control the circulating module 30 to initiate an automatic cleaning process, wherein the automatic cleaning process includes executing at least one disinfecting step and at least one cleansing step. The disinfecting step includes transmitting the solution of the source of first solution supply S1 to the receiving tank 20 to allow the tube body E2 being soaked into the solution outputted from the source of first solution supply S1, and then discharge the solution in the receiving tank 20 out of the receiving tank 20. The cleansing step includes transmitting a solution of the source of second solution supply S2 to the receiving tank 20 to allow the tube body E2 being soaked into the solution outputted from the source of second solution supply S2, and then discharge the solution in the receiving tank 20 out of the receiving tank 20.

For example, after the user conduct the step S101, the user could press the power button 421 on the operating panel 42 to power up the endoscope cleaning device 1, and then press the initiate button 422 of the operating panel 42 to drive the endoscope cleaning device 1 to automatically conduct the automatic cleaning process. In the current embodiment, the endoscope cleaning device 1 carries on the disinfecting step once at first. More specifically, a predetermined volume of a disinfection solution of the source of first solution supply S1 is transmitted to the receiving tank 20 by the first pump 31 through the tube T1 to allow the tube body E2 to be soaked in disinfection solution for a first predetermined time, and then the second pump 32 pumps the disinfection solution in the receiving tank 20 to the first storage tank 35 via the tube T3, thereby allowing the disinfection solution completely discharged from the receiving tank 20. After that, the endoscope cleaning device 1 carries on the cleansing step once. More specifically, a predetermined volume of the water of the source of second solution supply S2 is transmitted to the receiving tank 20 by the third pump 33 via the tube T2 to allow the tube body E2 to be soaked in the water outputted from the source of second solution supply S2 for a second predetermined time, and then the fourth pump 34 pumps the water in the receiving tank 20 to the second storage tank 36, thereby allowing the water to be complete discharged from the receiving tank 20. Finally, the endoscope cleaning device 1 carries on the cleansing step again to ensure that pollutants, contaminants, and the disinfection solution remained on the tube body E2 could be completely cleaned or washed away. It's worthy to mention that when the user press the initiate button 422 on the operating panel 42, the display screen 70 shows a time that needs for completing the automatic cleaning process and starts to countdown the time, so that the user could realize the time that needs for completing the automatic cleaning process from reading the time shown on the display screen 70. Additionally, in the current embodiment, the endoscope cleaning device 1 includes a level sensor 90 which is connected to the receiving tank 20. Before either the disinfecting step or the cleansing step, the level sensor 90 senses a liquid level of the solution in the receiving tank 20. When the liquid level of the solution in the receiving tank 20 is higher than a detecting position of the level sensor 90, the first pump 31 or/and the third pump 33 is prohibited to pump the solution of source of first solution supply S1 or the solution of the source of second solution supply S2 to the receiving tank 20, thereby preventing the solution from overflowing the receiving tank 20.

In the current embodiment, the automatic cleaning process sequentially conducts the disinfecting step once and the cleansing step twice as an example. In other embodiment, the automatic cleaning process could include the disinfecting step and the cleansing step once or more than once. Besides, the sequence of the disinfecting step and the cleansing step does not limited to conduct the disinfecting step first, and then conduct the cleansing step. For example, the automatic cleaning process could sequentially conduct the cleansing step once, the disinfecting step once, and cleansing step once.

Furthermore, the cleaning method of the endoscope includes operating the operating module 40 to control the circulating module 30 to initiate a compulsorily draining disinfecting solution step. The compulsorily draining disinfecting solution step includes draining the solution in the receiving tank 20 out of the receiving tank 20. For example, when the first pump 31 transmits the disinfection solution of the source of first solution supply S1 to the receiving tank 20 via the tube T1, the user could press the button 423 for compulsorily draining disinfecting solution on the operating panel 42 to stop the first pump 31 and initiate the second pump 32 to drain the disinfection solution in the receiving tank 20 to the first storage tank 35, thereby completely draining out the disinfection solution from the receiving tank 20. Besides, when the third pump 33 transmits the water of the source of second solution supply S2 to the receiving tank 20 via the tube T2, the user could press the button 424 for compulsorily draining water on the operating panel 42 to control the fourth pump 34 to pump the water in the receiving tank 20 to the second storage tank 36 through tube T4, thereby completely draining the water in the receiving tank 20.

The cleaning method of the endoscope includes at least one predetermined time to control an activation of the ultrasonic vibrator 80, wherein the at least one predetermined time includes a first predetermined time or a second predetermined time. In other words, when the tube body E2 is soaked in the disinfection solution or the water, the ultrasonic vibrator 80 could apply an ultrasonic vibration to the solution in the receiving tank 20, thereby sufficiently and completely cleaning, disinfecting, and sterilizing the outer surface and the inner surface of the tube body E2.

In view of above, by using the endoscope cleaning device and the cleaning method of the endoscope of the embodiment according to the present invention, the endoscope cleaning device could automatically conduct the automatic cleaning process to make the cleaning process of the endoscope be standardized and simplified, thereby enhancing the cleaning efficiency and cleaning quality.

It must be pointed out that the embodiment described above is only a preferred embodiment of the present invention. All equivalent structures and methods which employ the concepts disclosed in this specification and the appended claims should fall within the scope of the present invention.

What is claimed is:

1. An endoscope cleaning device applied to an endoscope, wherein the endoscope comprises a main body and a tube body which is connected to the main body; the endoscope cleaning device comprises:
a frame;
a receiving tank disposed on the frame and adapted to receive the tube body of the endoscope;
a circulating module connected to a source of first solution supply, a source of second solution supply, and the receiving tank;
an operating module disposed on the frame and electrically connected to the circulating module, wherein the operating module is provided for a user to control the circulating module to respectively transmit a solution provided from the source of first solution supply and a solution provided from the source of second solution supply to the receiving tank, and to drain the solution in the receiving tank out of the receiving tank.

2. The endoscope cleaning device as claimed in claim 1, wherein the circulating module comprises a first pump, a second pump, a third pump, a fourth pump, a first storage tank, and a second storage tank; the first pump, the second pump, the third pump, and the fourth pump are disposed to the frame and are controlled by the operating module, wherein the first pump is adapted to transmit the solution of the source of first solution supply to the receiving tank, the second pump is adapted to transmit the solution provided from the source of first solution supply to the first storage tank; the third pump is adapted to transmit the solution of the source of second solution supply to the receiving tank; the fourth pump is adapted to transmit the solution provided from the source of second solution supply to the second storage tank.

3. The endoscope cleaning device as claimed in claim 1, wherein the receiving tank is long cylindrical, the receiving tank has an opening on top; a minimum distance between the opening and a bottom of the receiving tank is greater than a length of the tube body.

4. The endoscope cleaning device as claimed in claim 3, further comprising a boot end positioning member, wherein the boot end positioning member is located above the receiving tank, and the boot end positioning member has a through hole; the tube body of the endoscope passes through the through hole, and the main body of the endoscope abuts against a side of the boot end positioning member.

5. The endoscope cleaning device as claimed in claim 4, wherein the receiving tank has a longitudinal axis which is parallel to a length direction of the receiving tank, and the longitudinal axis passes through the opening on top of the receiving tank and the through hole of the boot end positioning member.

6. The endoscope cleaning device as claimed in claim 5, wherein the receiving tank comprises a tank body, a top lid, and a bottom lid; the top lid and the bottom lid are detachably engaged with the tank body; the top lid has the opening and two perforations; the bottom lid has two perforations; one of the two perforations of the top lid is connected to the source of first solution supply via a tube, and the other one of the two perforations of the top lid is connected to the source of second solution supply via another tube; one of the two perforations of the bottom lid is connected to the first storage tank via a tube, and the other one of the two perforations of the bottom lid is connected to the second storage tank via another tube.

7. The endoscope cleaning device as claimed in claim 5, further comprising a cord end positioning member, wherein endoscope cord end positioning member is disposed on the frame and is located above the receiving tank, the main body of the endoscope has a boot end and a cord end; the cord end positioning member is adapted to receive the cord end of the main body; when the boot end of the main body abuts against the side of the boot end positioning member, and the cord end of the main body is disposed in the cord end positioning member, the cord end positioning member restricts the main body to rotate about the longitudinal axis.

8. The endoscope cleaning device as claimed in claim 1, further comprising an ultrasonic vibrator disposed in the receiving tank.

9. The endoscope cleaning device as claimed in claim 1, wherein the source of first solution supply is a disinfection solution supply source, and the source of second solution supply is a water supply source.

10. A cleaning method of an endoscope by using the endoscope cleaning device as claimed in claim 1, wherein the cleaning method comprises:
A. putting the tube body of the endoscope into the receiving tank; and
B. manipulating the operating module to control the circulating module to initiate an automatic cleaning process, wherein the automatic cleaning process comprises at least one disinfecting step and at least one cleansing step in order
wherein the at least one disinfecting step comprises transmitting a solution of the source of first solution supply to the receiving tank to allow the tube body to be soaked into the solution outputted from the source of first solution supply, and then discharge the solution in the receiving tank out of the receiving tank;
wherein the at least one cleansing step comprises transmitting a solution of the source of second solution supply to the receiving tank to allow the tube body to be soaked into the solution outputted from the source of second solution supply, and then discharge the solution in the receiving tank out of the receiving tank.

11. The cleaning method as claimed in claim 10, wherein the at least one disinfecting step allows the tube body to be soaked in the solution outputted from a first predetermined time, and the at least one cleansing step allows the tube body to be soaked in the solution outputted from the source of second solution supply for a second predetermined time.

12. The cleaning method as claimed in claim 10, wherein the circulating module of the endoscope cleaning device comprises a first pump, a second pump, a third pump, a fourth pump, a first storage tank, and a second storage tank; the first pump, the second pump, the third pump, and the fourth pump are disposed to the frame; the cleaning method comprises controlling the first pump to transmit a predetermined volume of the solution of the source of first solution supply to the receiving tank and controlling the third pump to transmit a predetermined volume of the solution of the source of second solution supply to the receiving tank.

13. The cleaning method as claimed in claim 11, wherein the endoscope cleaning device comprises an ultrasonic vibrator disposed in the receiving tank; the cleaning method comprises controlling the ultrasonic vibrator to initiate at either the first predetermined time or the second predetermined time.

14. The cleaning method as claimed in claim 13, wherein the source of first solution supply is a disinfection solution supply source, and the source of second solution supply is a water supply source.

15. The cleaning method as claimed in claim 10, comprising operating the operating module to control the circulating module to initiate a compulsorily draining disinfecting solution step, wherein the compulsorily draining disinfecting solution step comprises draining the solution in the receiving tank out of the receiving tank.
